SEQUENCE LISTING (3 sequences)

SEQ ID NO: 1 - protein, 30 aa, Homo Sapiens
  ProANF 1-30 First 30 amino acids from N-terminus of: IDENTIFICATION METHOD: Infusion of peptide into animals and humans.; Lowers blood pressure, causes a diuresis and enhances sodium excre
  description: Peptide consisting of 30 amino acids constituting N- terminal 30 amino ac
  hypothetical: No
  anti-sense: No
  fragment type: N-terminal fragment
  original source: Genomic mRNA
  original source tissue type: Heart muscle
  original source cell type: myocyte
  chromosome/segment: Distal short arm of Chromosome 1 in band 1 P36
  map position: band 1
  units: P36 atrial natriuretic factor prohormon
  authors: Vesely, David L, Douglass, Margaret A, Dietz, John R, Gower, William R Jr, M
  title: Three peptides from atrial natriuretic factor prohormone amino terminus lower
  journal: Circulation
  volume: 90
  issue: 3
  pages: 1129-1140
  date: September, 1994
  relevant residues in seq id no: 30
NPMYNAVSNADLMDFKNLLDHLEEKMPLED

SEQ ID NO: 2 - protein, 37 aa, Homo Sapiens
  ProANF 31-67 37 amino acids from middle of atrial: IDENTIFICATION METHOD: Infusion of peptide into animals and humans.; Lowers blood pressure, causes a diuresis and enhances sodium excre
  description: Peptide consisting of 37 amino acids originating in middle of 126 amino
  hypothetical: No
  anti-sense: No
  fragment type: middle fragment of ANF prohormone
  original source: Genomic mRNA
  original source tissue type: Heart muscle
  original source cell type: myocyte
  chromosome/segment: Distal short arm of Chromosome 1 band 1 P36
  map position: band 1
  units: P36 natriuretic factor prohormone consisti
  authors: Vesely, David L, Douglass, Margaret A, Dietz, John R, Gower, William R Jr, M
  title: Three peptides from atrial natriuretic factor prohormone amino terminus lower
  journal: Circulation
  volume: 90
  issue: 3
  pages: 1129-1140
  date: September, 1994
  relevant residues in seq id no: 37
EVVPPQVLSEPNEEAGAALSPLPEVPPWTGEVSPAQR

SEQ ID NO: 3 - protein, 20 aa, Homo Sapiens
  ProANF 79-98 20 amino acids from middle of atrial: IDENTIFICATION METHOD: Infusion of peptide into animals and humans.; Lowers blood pressure, causes a diuresis and enhances potassium ex
  description: Peptide consisting of 20 amino acids (i.e., amino acids 79-98) from midd
  hypothetical: No
  anti-sense: No
  fragment type: middle fragment
  original source: Genomic mRNA
  original source tissue type: Heart muscle
  original source cell type: myocyte
  chromosome/segment: Distal short arm of Chromosome 1 band 1 P36
  map position: band 1
  units: P36 natriuretic factor prohormone consisti
  authors: Vesely, David L, Douglass, Margaret A, Dietz, John R, Gower, William R Jr, M
  title: Three peptides from atrial natriuretic factor prohormone amino terminus lower
  journal: Circulation
  volume: 90
  issue: 3
  pages: 1129-1140
  date: September, 1994
  relevant residues in seq id no: 20
SSDRSALLKSKLRALLTAPR

(19) United States
(12) Patent Application Publication
(10) Pub. No.: US 2006/0205642 A1
(43) Pub. Date: Sep. 14, 2006

(54) Oral methods of treatment using proANF peptides

(76) Inventor: David Lynn Vesely, Tampa, FL (US)

Correspondence Address: David L. Vesely, M.D Ph.D, 5020 Londonderry Drive, Tampa, FL, 33647, US

(21) Appl. No.: 11/074,063

(22) Filed: Mar. 8, 2005

(51) Int. Cl. A61K 38/18

(52) U.S. Cl. 514/12

(57) ABSTRACT

A method of treatment of hypertension, congestive heart failure, pulmonary edema, nephrotic syndrome, acute and chronic renal failure, toxemia of pregnancy, hepatic cirrhosis, and/or hyperkalemia. Humans or other mammals are administered an effective amount of peptide(s) consisting of amino acids 1-30 (proANF 1-30), amino acids 31-67 (proANF 31-67) and amino acids 79-98 (i.e., proANF 79-98) of the human sequence of the atrial natriuretic factor (ANF) prohormone. Pharmaceutical compositions include such peptides in an effective concentration within a pharmaceutically acceptable liquid or solid carrier given orally.

The present invention is directed to methods for pharmaceutical treatment of 1) congestive heart failure, including a severe form called pulmonary edema, nephrotic syndrome, renal failure, hepatic cirrhosis and toxemia of pregnancy each of which are characterized by retention of sodium and water and 2) treatment of high blood pressure and myocardial ischemia of mammals including humans with new peptides that increase sodium and water excretion and also lower blood pressure. One of these new peptides, i.e., proANF 79-98 has strong potassium excreting properties which direct it to the treatment of hyperkalemia (i.e., high blood potassium) associated with acidosis, digitalis overdose, succinylcholine, insulin deficiency, acute and chronic renal failure, Addison's disease (i.e., adrenal insufficency) and the syndromes of hyperkalemic periodic paralysis, and hyporeninemic hypoaldosteronism. Specifically, the invention is directed to methods of treatment of mammals (especially humans) by administration of pharmaceutical compositions containing one or more of three peptide hormones derived from the 98 amino acid amino terminal polypeptide of the human sequence of 126 amino acid atrial natriuretic peptide prohormone (hereafter referred to as proANF).

### BACKGROUND OF THE INVENTION

DeBold et al. in Life Sci. 28, 89-94, (1981) demonstrated that crude rat heart atrium extracts increased natriuresis and lowered blood pressure when intravenously injected into other rats which suggested that the heart made one or more blood pressure and/or sodium excreting hormones. This substance(s) was called atrial natriuretic factor (ANF) since 1) the substance was obtained from the atrium of the heart and had more biological activity than extracts of the ventricle of the heart, 2) it caused a natriuresis (i.e., enhanced sodium excretion) and 3) it was unknown at that time that the factor was a peptide (s) (DeBold et al., ibid).

The amino acid sequence of the polypeptide prohormone containing the biologically active hormones was then determined for rat (Yamanaka et al., Nature 309:719-722, 1984; Maki et al., Nature 309:722-724, 1984; Zivin et al., Proc. Natl. Acad. Sci. 81:6325-6329, 1984; Seidman et al., Science 225:324-326) and humans (Oikawa et al., Nature 309:724-726, 1984; Zivin et al., ibid). It was found that the amino acids (a.a.) making up the peptides derived from the rat and human atrial natriuretic factor prohormones were different. Further investigation revealed that the ANF prohormone is synthesized within the atrial myocyte as a 151 amino acid (a.a.) preprohormone. This preprohormone is processed within the endoplasmic reticulum of humans to form a prohormone consisting of 126 a.a. after removal of the 25 a.a. signal peptide from its N-terminal end. This 126 a.a. ANF prohormone is the main storage form of atrial natriuretic peptides within the heart (and within other tissues).

At first it was thought that only the C-terminus (i.e., ANF) of ANF prohormone circulated. Before any portion of the rest of the prohormone was known to circulate Dr. Frossman patented the whole rat prohormone which he called Cardiodilatin and portion(s) thereof (U.S. Pat. No. 4,751,284). In this patent Dr. Frossman patented the rat sequence of the ANF prohormone. In his patent he stated that parts of the whole prohormone had activity if the C-terminal end (i.e., ANF) was attached to it. In publications he has stated that the rest of the rat prohormone only has activity if the C-terminus (i.e., ANF with its intact ring structure) of the rat prohormone is attached to it (Frossman W. G., Contr. Nephrol. 50:1-13, 1986) (see Attachment A). Dr. Frossman has never published that other peptide(s) originating from the rat ANF prohormone that he patented have any biologic activity by themselves. Dr. Frossman has further stated on page 11 of Attachment A “I think that the 28 a.a. C-terminal is sufficient for explaining all effects and that an N-terminal fragment does not qualitatively affect the biologic activity . . . the synthetic N-terminal segments that we have investigated so far do not exhibit the biologic activity of cardiodilatin”. It has been over 20 years since Frossmann submitted his patent and he has never published that any portion of cardiodilatin (i.e., rat ANF prohormone) has any biologic activity if the respective portion is not attached to the ANF portion of cardiodilatin.

On the other hand, my Method-of-Use patent is for peptides consisting of amino acids 1-30, 31-67, and 79-98 of the human ANF prohormone (see table 1 for amino acid sequence of human ANF prohormone) which have marked blood pressure lowering, sodium and/or potassium excreting properties in both healthy animals (i.e., rats, Martin et al., Am. J. Physiol. 258:F1401-1408, 1990; Attachment B) and humans (Vesely et al., Circulation 90:1129-1140, 1994; Attachment C). This Method-of-Use application is, thus, for peptides with distinctly different amino acid sequences and also distinctly different biologic properties than rat cardiodilatin and fragments thereof patented by Frossmann. The peptides from the human sequence have potent biologic properties. This Method-of-Use application is not for the rat forms of proANF 1-30, proANF 31-67 or proANF 79-98 but rather the human forms which I discovered have biologic effects in both animals and humans (see Attachments B & C).

In addition to the human forms of these peptides having biologic potential in humans as therapy as detailed below, these peptides consisting of amino acids 1-30 (tentatively named long acting natriuretic peptide), amino acids 31-67 (tentatively named vessel dilator) and amino acids 79-98 (tentatively named kaliuretic peptide) are actually peptide hormones since they circulate in humans (Vesely et al., Circulation 90:1129-1140, 1994; Attachment C; Gower et al., Peptides 15:861-867, 1994) and have biologic effects in other organs (i.e., target tissues) different from the organ or gland from which they are released (Vesely et al., Circulation 90:1129-1140, 1994).

As one observes in FIGS. 1-4, the above enumerated peptide hormones have significant blood pressure lowering, diuretic, sodium and/or potassium excreting properties in healthy humans. These peptide hormones also have these significant properties for treating persons with congestive heart failure (FIGS. 5,6). It has further been demonstrated that in humans that none of the above delineated peptide hormones work through ANF (i.e., the C-terminus of the ANF prohormone, Vesely et al., J. Clin. Endo. and Metab. 78:1128-1134, 1994; Attachment D).

When one examines the amino acids of the rat prohormone in the Frossmann U.S. Pat. No. 4,751,284, one notes the amino acid sequence of the peptides are distinctly different beginning as early as the 3rd amino acid in the prohormone with his patented sequence being Val-Try-Gly-Ser while the same amino acid sequence in the human prohormone is Met-Try-Asn-Ala as in table 1. The presently described human peptides are, thus, distinctly different peptides from those Dr. Frossmann has patented. The changing of a single amino acid in a specific peptide can have profound effects on biologic activity. Thus, the peptides in his patent with markedly different amino acids sequences might be expected to have a difference in biologic properties or no biologic effects. None of the human peptides in the present Method-of-Use application have a ring structure within their peptide sequence while all of the peptides that Frossmann has stated that have activity in Frossmann's patent are characterized by having a ring structure. This ring structure is thought to be essential for ANF and extended ANF peptides to bind to their receptor while human proANF 1-30 and 31-67 bind to separate and distinct receptors from the ANF receptor(s) (Vesely et al., Peptides 11;193-197, 1990; Vesely et al., J. Clin. Endocrinol. Metab. 71:1138-1146, 1990). The peptides proposed for Method-of-Use in the present patent application are linear peptides. They do not have a ring structure. Further, the human proANF's 1-30, 31-67, and 79-98 in addition to having distinct receptors from ANF also have different mechanisms of action from ANF (Chiou and Vesely: Endocrinology, 136:2033-2039, 1995; Attachment E).

### SUMMARY OF INVENTION

In vivo animal (Attachment B) and human (Attachment C) testing demonstrate that potent natriuretic, diuretic, and blood pressure reducing effects are exhibited by two peptide hormones originating from the human atrial natriuretic factor (ANF) prohormone consisting of amino acid 1-30 and 31-67 of the human prohormone while another peptide hormone consisting of amino acids 79-98 of the human ANF prohormone has diuretic, kaliuretic (i.e., potassium excreting) and blood pressure lowering properties. (See Table 1 for sequences).

TABLE I

Amino acid sequence of human ANF prohormone
polypeptide

H₂N-Asn-Pro-Met-Tyr-Asn-Ala-Val-Ser-Asn-Ala-Asp-
Leu-Met-Asp-Phe-Lys-Asn-Leu-Leu-Asp-His-Leu-Glu-
Glu-Lys-Met-Pro-Leu-Glu-Asp-Glu-Val-Val-Pro-Pro-
Gln-Val-Leu-Ser-Glu-Pro-Asn-Glu-Glu-Ala-Gly-Ala-
Ala-Leu-Ser-Pro-Leu-Pro-Glu-Val-Pro-Pro-Trp-Thr-
Gly-Glu-Val-Ser-Pro-Ala-Gln-Arg-Ser-Ser-Asp-Arg-
Ser-Ala-Leu-Leu-Lys-Ser-Lys-Leu-Arg-Ala-Leu-Leu-
Thr-Ala-Pro-Arg-Ser-Leu-Arg-Arg-Ser-Ser-Cys-Phe-
Gly-Gly-Arg-Met-Asp-Arg-Ile-Gly-Ala-Gln-Ser-Gly-
Leu-Gly-Cys-Asn-Ser-Phe-Arg-Tyr-COOH

The carboxy terminal 28 amino acid fragment of the pro-ANF prohormone (amino acids 99-126 as shown in Table 1) has been termed alternatively cardioatrin, atrial natriuretic peptide (ANP), and atrial natriuretic factor (ANF).

Clinical trials in healthy human subjects have demonstrated that these peptides have potent diuretic, blood pressure lowering, natriuretic and/or kaliuretic effects (FIGS. 1-4; Attachment C). In humans as well as in other mammals proANF 1-30 and 31-67 have unique sodium excreting properties compared to ANF with all of ANF's sodium excreting effects ceasing within 30 minutes while proANF 1-30 and 31-67's sodium excreting effects were significantly (P<0.001) prolonged compared to ANF (FIG. 3). ProANFs 1-30, 31-67, and 79-98 have significant diuretic properties in humans increasing urine flow 4-12-fold and this increased urine flow was still significantly increased 2-3 hours after stopping their infusions, which also is markedly prolonged compared to ANF (FIG. 2). ProANF 79-98 has the unique property of being the strongest stimulator of potassium excretion of any of the atrial peptides and (FIG. 4) should be useful for treating states associated with hyperkalemia. Each of 3 peptides of the instant invention have significantly prolonged half-lives compared to ANF (Ackermann et al., J. Clin. Pharmacol. 32:415-421, 1992) which in addition to their unique and prolonged effects compared to ANF would cause them to be given less frequently in treatment (and therefore preferred) of conditions characterized by high blood pressure, sodium and water retention or hyperkalemia that were enumerated above.

I have now infused the peptides of this invention into over 50 humans with congestive heart failure. ProANFs 1-30, 31-67 and 79-98 cause a marked diuresis in humans with congestive heart failure which is significant (P<0.01) for at least 3 hours after stopping their respective infusions (FIGS. 5,6; see references below). The persons with congestive heart failure reported that they felt better after the infusion of these peptides. The instant invention is shown herein to be reduced to therapeutic use in humans with congestive heart failure. Each of these peptide hormones have been give to humans with congestive heart failure with beneficial effects to these persons. ProANF 31-67 for instance has significant beneficial diuretic and natriuretic effects in persons with congestive heart failure which are not blunted (i.e., decreased) compared to healthy individuals (FIG. 5; Vesely D L, et al. Circulation 98:323-329, 1998; Attachment F). Vessel dilator also has the beneficial effects of decreasing systemic vascular resistance, pulmonary vascular resistance, pulmonary capillary wedge pressure, and central venous pressure while increasing cardiac output, cardiac index and stroke volume in persons with congestive heart failure (FIG. 6). Vessel dilator does not cause any significant change in heart rate in persons with congestive heart failure (FIG. 6). Likewise, proANF 1-30 has significant effects in humans with congestive heart failure (Vesely D L, et al. Am Heart J 138:625-632, 1999; Attachment G). ProANF 79-98 also has beneficial diuretic and natriuretic effects in persons with congestive heart failure (Nasser A, et al. Am J Cardiol 88:23-29, 2001; attachment H). ProANF 79-98, which does not have natriuretic effects in healthy humans, has natriuretic effects in persons with congestive heart failure (ibid).

ProANF 31-67 improves acute tubular necrosis and renal failure even when renal failure has been established for two days before proANF 31-67 is administered (Clark L C, et al., Am. J. Physiol. 278:H1555-H1564, 2000; Exhibit I). Twenty-four Sprague-Dawley rats had ischemic nonoliguric acute renal failure (ARF) induced by vascular clamping resulting in their preischemic blood urea nitrogen (BUN) and creatinine levels of 16±1 and 0.56±0.05 mg/dl to increase to 162±4 and 8.17±0.5 mg/dl, P<0.001, respectively, at day 4 of postischemia (n=17) (ibid). ProANF 31-67 (0.3 μg·kg⁻¹·min⁻¹ intraperitoneally) decreased the BUN and creatinine levels to 53±17 mg/dl and 0.98±0.12 mg/dl (P<0.001) in seven animals where ARF had been established for two days. Water excretion doubled with ARF and was further augmented by proANF 31-67. Transthoracic echocardiography revealed left ventricular dilation as a probable cause of the increase in proANF 31-67 in the circulation with ARF and vessel dilator infusion reversed this dilation (ibid). At day 6 of ARF, mortality decreased to 14% with proANF 31-67 form 88% without proANF 31-67 (FIG. 7). Acute tubular necrosis was <5% in the proANF 31-67-treated ARF animals compared with 25% to >75% in the placebo-treated animals (ibid). Conclusion: ProANF 31-67 improves acute tubular necrosis and renal function in established ARF.

The present invention provides method(s)-of-treatment which use these three 100% pure peptides which are sequenced with an peptide sequencer (and are not derived from natural products such as using heart tissues to isolate these peptides). These peptides are not known to exist in the forms which I am seeking to patent in natural products such as heart or any other animal or plant tissues. In tissues the whole 126 amino acid prohormone is present but the individual distinct peptides proANF 31-67 and proANF 79-98 are not. Pharmaceutical compositions in accordance with the present invention include one or more of the three amino-terminal pro-ANF peptides, or non-toxic salts thereof, dispersed in a pharmaceutically acceptable liquid or solid carrier. The administration of one or more of these peptides, or pharmaceutically acceptable addition of salts thereof, to mammals (especially humans) in accordance with the invention may be carried out for treatment of hypertension, disorders of water and sodium metabolism, (delineated above), hyperkalemia, or to counteract development of congestive heart failure and/or renal failure; in addition, pharmaceutical compositions containing any of the three peptides may be used as a smooth muscle relaxant. These three peptides mechanism of action of causing a natriuresis involves the enhancement of synthesis of prostaglandin E₂ (FIG. 8) which, in turn, inhibits Na⁺K⁺-ATPase in the kidney (FIG. 9).

### BRIEF DESCRIPTION OF THE DRAWINGS

FIG. 1. Illustrates the data with respect to human atrial natriuretic factor prohormone peptides (proANFs) 1-30, 31-67, 79-98, and atrial natriuretic factor (ANF) decreasing systolic and diastolic blood pressures in healthy human volunteers. The decreases in both systolic and diastolic blood pressures secondary to proANFs 1-30, 31-67, 79-98, and ANF were significant at the P<0.05 level when evaluated by paired t test. Values are mean±SEM of 6 healthy subjects for each peptide.

FIG. 2. Plot shows influence of human atrial natriuretic factor prohormone peptides 1-30, 31-67, 79-98 and atrial natriuretic factor (ANF) on urine flow in healthy human volunteers. Individual subjects (n=6 for each peptide) received 100 ng/kg body wt per minute of each of the respective peptides for 1 hour by intravenous infusion. The increase in urine volume (in milliliters) secondary to each of the respective peptides was significant at P<0.05 when evaluated by ANOVA. *Time points at which urine flow was significantly (P<0.05) increased compared with baseline urine flow in the same subjects when evaluated by repeated measures of analysis of variance(ANOVA). Mean±SEM values for each peptide at each point are illustrated.

FIG. 3. Plot shows effect of human atrial natriuretic factor prohormone peptides (proANFs) 1-30, 31-67, 79-98, and atrial natriuretic factor (ANF) on sodium excretion in healthy human volunteers. When infused at their 100 ng/kg body wt concentration per minute for 60 minutes, each of these respective peptides except proANF 79-98 significantly (P<0.05) increased sodium excretion compared to baseline sodium excretion in the same subjects when evaluated by repeated-measures of ANOVA. Values are mean±SEM values of 6 healthy subjects for each group.

FIG. 4. Illustrates the data with respect to human atrial natriuretic factor prohormone peptides (proANFs) 1-30, 31-67, 79-98, and atrial natriuretic factor (ANF) on potassium excretion in healthy humans. Values are mean±SEM of 6 healthy subjects for each peptide. The enhancement of potassium excretion by proANFs 1-30, 79-98, and ANF was significant (P<0.05) when infused at their 100 ng/kg body wt concentrations when evaluated as a group by ANOVA.

FIG. 5. Vessel dilator increases the fractional excretion of sodium (FE_{Na}) in persons with congestive heart failure. *Time points when filtration fraction of sodium was significantly (P<0.05) increased secondary to vessel dilator infusion at its 100 ng/kg body weight per minute concentration for 60 minutes when evaluated by repeated-measures ANOVA (n=6 for each).

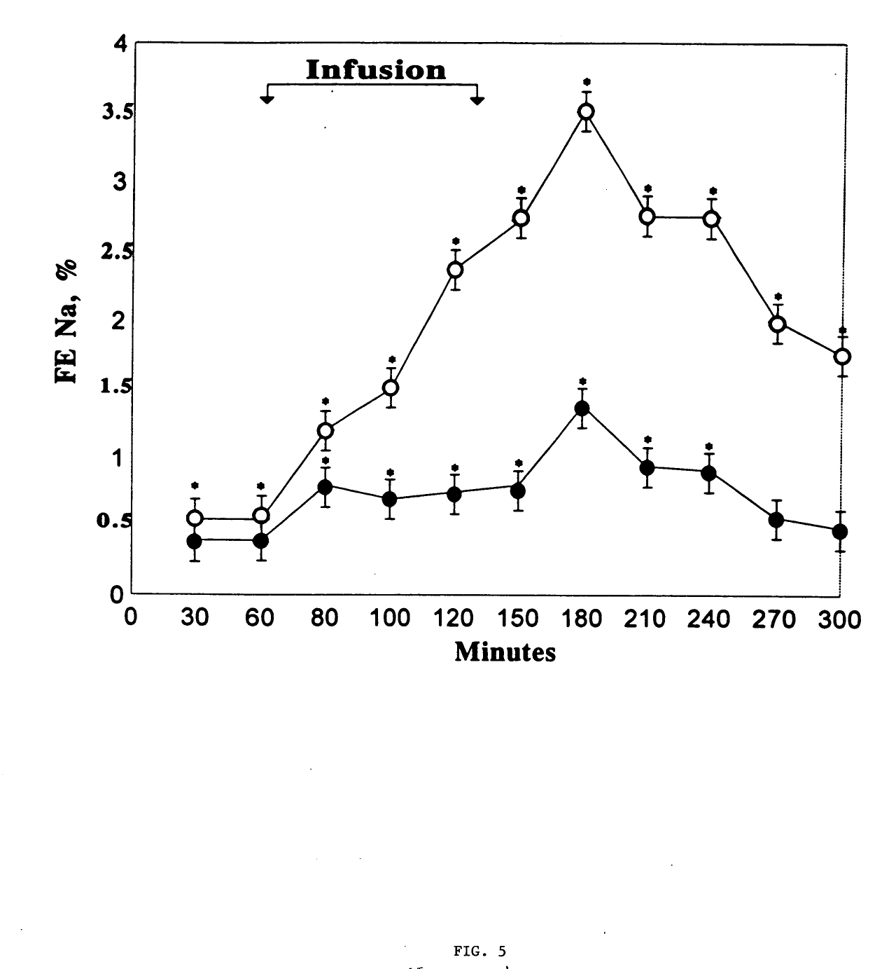

FIG. 6. Systemic vascular resistance (SVR), pulmonary vascular resistance (PVR), pulmonary capillary wedge pressure (PWP), and central venous pressure (CVP) decrease secondary to vessel dilator. Each of these decreases were significant at P<0.05 when evaluated by repeated-measures ANOVA, whereas no significant changes were found in heart rate (HR) or pulmonary artery pressure (PAP). Cardiac output (CO), cardiac index (CI), and stroke volume index (SVI) were significant (P<0.05) increased when evaluated by repeated-measures ANOVA. There was no change in any of these parameters in the congestive heart failure subjects who received vehicle only (n=4 for each group).

FIG. 7. Kaplan-Meir plot of survival. There was a significant advantage with respect to survival in vessel dilator-treated acute renal failure (ARF) rats (●) (n=6) from day 4 (P<0.01) and day 5 onward (P<0.001) compared with placebo-treated ARF rats (◯) (n=17). *Groups were significantly different.

FIG. 8. ProANF 79-98 (kaliuretic peptide), proANF 1-30 (long acting natriuretic peptide), and proANF 31-67 (vessel dilator) each increase prostaglandin E₂ (PGE₂) synthesis. The increase in PGE₂ synthesis by each of these peptides was significant at P<0.05, whereas atrial natriuretic factor (ANF) had no significant effect on PGE₂ synthesis when evaluated by Analysis of Variance followed by Duncan's Multiple Range Test (MRT) (n=6)

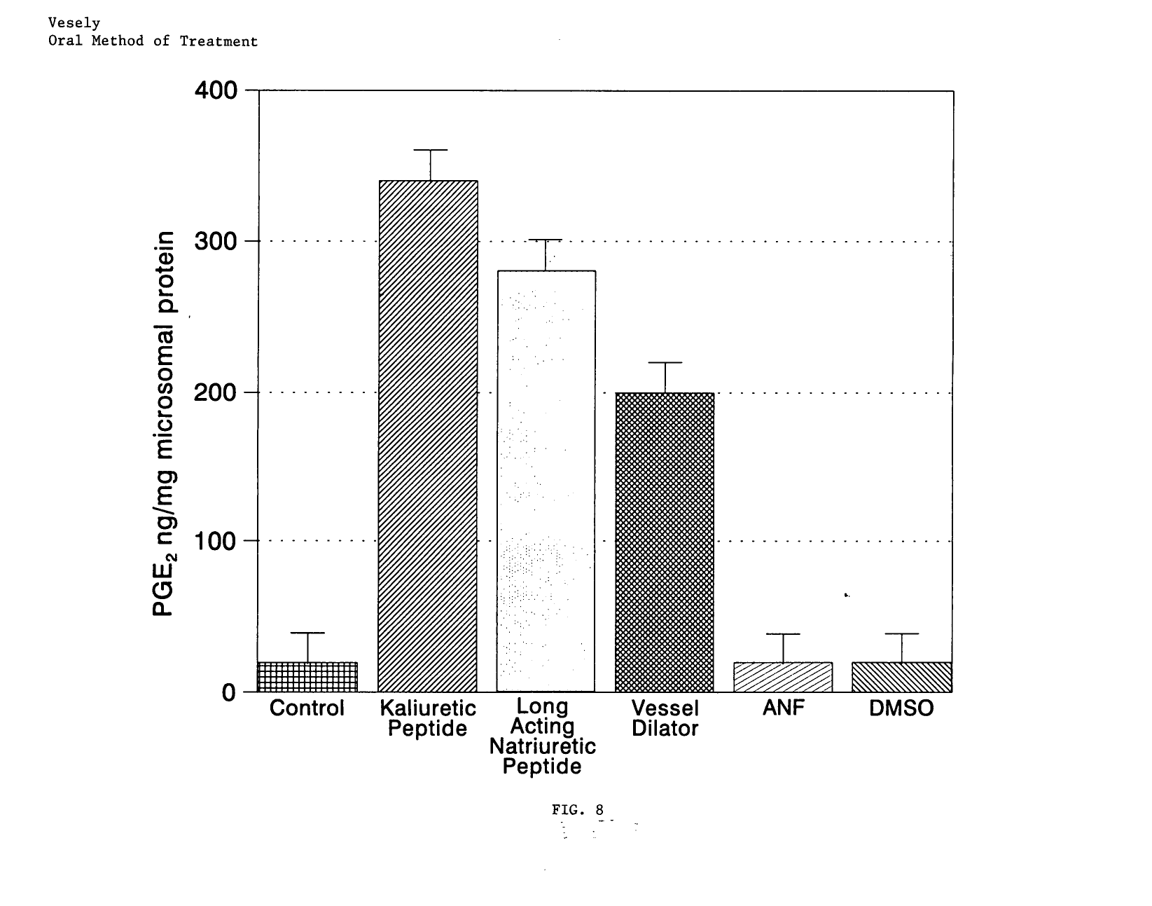

FIG. 9. Inhibition of renal medullary Na+-K+-ATPase activity by proANF 79-98 (kaliuretic peptide), proANF 1-30 (long acting natriuretic peptide), proANF 31-67 (vessel dilator), and angiotensin II at their 10¹¹ M concentrations. Atrial natriuretic factor (ANF) (10¹¹ M) did not significantly inhibit Na+-K+-ATPase when evaluated by Analysis of Variance (ANOVA), whereas each of the other peptides inhibition of Na+-K+-ATPase was significant at P<0.01 (*). Naproxen (0.5 mM), a prostaglandin synthesis inhibitor, completely blocked each of these peptide' effects on renal medullary Na+-K+-ATPase with its effect being significantly different (P<0.001) from the effects of each of the peptides (except for ANF, which had no effect by itself) when evaluated by ANOVA followed by Duncan's Multiple Range Text (MRT) (n=6 for each group).

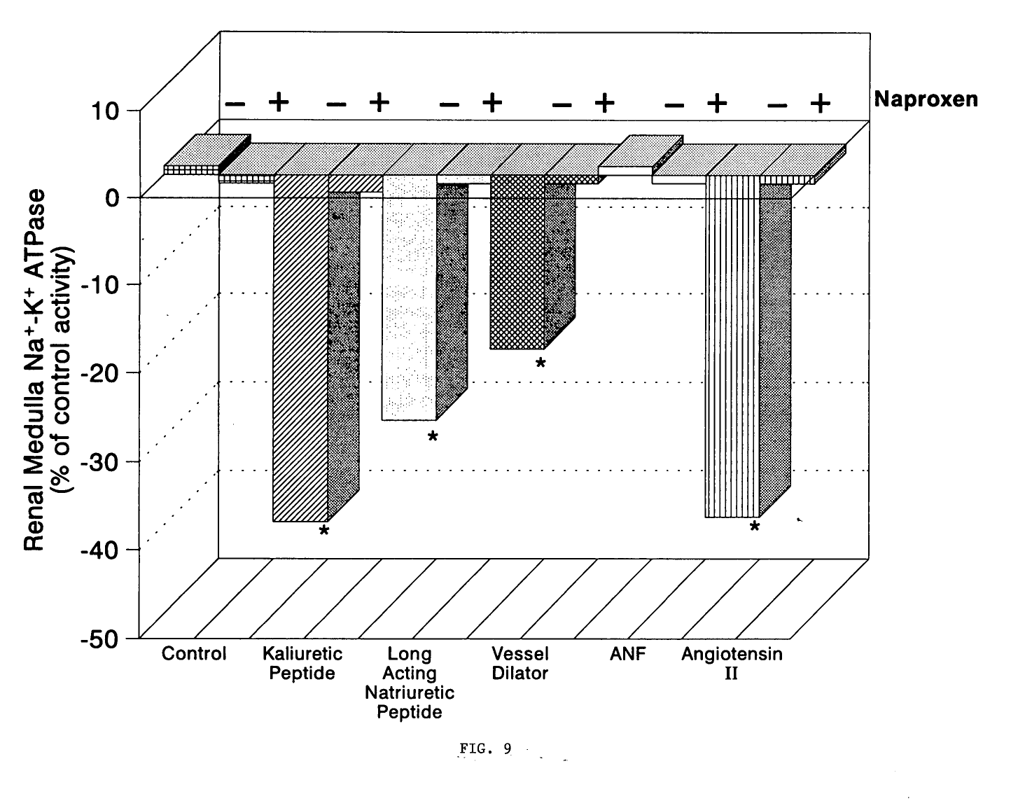

### DETAILED DESCRIPTION OF THE INVENTION

The nomenclature used to define the peptides is that specified by Schroder & Luhke, “The Peptides,” Academic Press (1965) wherein, in accordance with conventional representation, the amino group appears to the left and the carboxy group to the right. Where the amino acid residue has isomeric forms, it is the L-form of the amino acid that is represented. The invention provides pharmaceutical compositions containing one or more of the peptides originating from the human ANF prohormone consisting of amino acids 1-30, 31-67, 79-98, of this prohormone, i.e., pro-ANF (1-30), pro-ANF (31-67) and pro-ANF (79-98), which have the sequences shown in Table I.

These three peptides may be synthesized by the following suitable techniques: solid-phase techniques, by partial solid-phase techniques, by fragment condensation, by classical solution addition or by an automated 8-channel peptide sequencer which can synthesize up to 8 different 20 residue peptides in 2 days. Examples of the techniques of exclusively solid-state synthesis are set forth in the textbook “Solid-Phase Peptide Synthesis,” (Stewart & Young, Freemon & Co., San Francisco, 1969); and examples are the disclosures of U.S. Pat. Nos. 3,842,067 and 3,862,925. Such human peptides are currently being sequenced commercially by automated peptide sequencers from laboratories such as Peninsula Laboratories, Belmont, Calif. Synthesis by the use of recombinant DNA techniques may also be used by suitably employing a structural gene coding for the desired form of the peptide. The synthetic peptides may be obtained by transforming a microorganism, either a procaryote or eucaryote, such as yeast, using an expression vector including a promoter and operator together with such a structural gene and causing such transformed microorganism to express the peptide. A non-human animal may also be used to produce the peptide by gene-farming using such a structural gene or by using microinjection of embryos; the synthetic peptide is then suitably recovered from the animal by extraction from sera or the like.

The following examples demonstrate the therapeutic use fullness of the three human pro-ANF peptides.

### EXAMPLE I

Pure synthetic human sequences of the following three human pro-ANF peptides and ANF are tested for vasodilation of porcine aortas (with or without the endothelium present): human pro-ANF 1-30 having the formula H₂N-Asn-Pro-Met-Tyr-Asn-Ala-Val-Ser-Asn-Ala-Asp-Leu-Met-Asp-Phe-Lys-Asn-Leu-Leu-Asp-His-Leu-Glu-Glu-Lys-Met-Pro-Leu-Glu-Asp-OH; pro-ANF 31-67 having the formula H-Glu-Val-Val-Pro-Pro-Gln-Val-Leu-Ser-Glu-Pro-Asn-Glu-Glu-Ala- Gly-Ala-Ala-Leu-Ser-Pro-Leu-Pro-Glu-Val-Pro-Pro-Trp-Thr-Gly-Glu-Val-Ser-Pro-Ala-Gln-Arg-OH; and pro-ANF (79-98) having the formula H-Ser-Ser-Asp-Arg-Ser-Ala-Leu-Leu-Lys-Ser-Lys-Leu-Arg-Ala-Leu-Leu-Thr-Ala-Pro-Arg-OH.

30 Kg pigs are sacrificed, and aortic strips are harvested. The strips are attached to F-60 transducer (Narco-BioSystems, Houston, Tex.) and attached to a physiography (Narco-BioSystems) while being submerged in a muscle tissue bath comprising a Krebs Ringer solution at 37° C., with 95% O₂-5% CO₂, bubbled through the bath. Percentage vasodilation is measured versus time after preconstriction with 0.3 micromolar phenylephrine. The three pro-ANF peptides show maximal vasodilation ten minutes after the peptides are added to the bath. Furthermore, the vasodilation by all four peptides originating from the ANF prohormone (pro-ANF 1-30, pro-ANF 31-67, pro-ANF 79-98, and ANF—also referred to herein as pro-ANF 99-126) is associated with a four-to-five fold increase in cyclic GMP levels over baseline levels. (Vesely et al., Biochem. Biophys. Res. Commun. 148:1540-1548, 1987).

To determine whether these peptides (pro-human (h)ANF 1-30, pro-hANF 31-67, and pro-hANF 79-98) do have direct effects on smooth muscle, these three pro-ANF fragments plus ANF were studied with cultured smooth muscle cells. All four peptides (pro-hANF 1-30, pro-hANF 31-67, pro-hANF 79-98, and pro-hANF 99-126) of the human sequence of the ANF prohormone increase cyclic GMP levels in the cultured smooth muscle cells, which is caused by activation of particulate guanylate cyclase (ibid). Dose response curves reveal that half maximal activation (ED₅o) occurs at a concentration of about 10 nanomolar and maximal activation of quanylate cyclase activity occurs at concentrations of about 1 μM for all four peptides in porcine aorta and in isolated smooth muscle cells. These data demonstrate that pro-hANF 1-30, pro-hANF 31-67 and pro-hANF 79-98 of the human ANF prohormone cause vasodilation and activate the guanylate cyclase-cyclic GMP system. The site of action in the aorta appears to be the smooth muscle cell and not the endothelium for all four of the peptide fragments of prohormone ANF since all four peptide's vasodilatory effects are equal with or without endothelium present (Vesely D L, et al., Biochem. Biophys. Res. Commun. 148:1540-1548, 1987).

### EXAMPLE II

The same three peptides and ANF from Example I are tested using the rat clearance model. Six control rats and six experimental Munich-Wistar rats, each weighing 150-200 grams, are used for each peptide, with catheters being placed in both jugular veins and in the femoral artery. The ureters are cannulated for simultaneous urine collections with femoral artery blood collections.

After inserting the catheters, a stabilization of one hour is allowed. A 10 μg/kg bolus of each of the respective human sequences of the peptides is given via the jugular vein to six experimental rats followed by a sustaining infusion of 0.1 μg/kg/min for 60 minutes. Blood and urine samples are collected at 20 minute intervals during this 60 minute infusion. Table II illustrates the effect of each of these peptides on diuresis and salt excretion. In both the control and experimental animals, hematocrits and plasma sodium values remained at the same levels pre- and post-infusion while blood pressure decreased with each of these peptides (see Attachment B).

TABLE II
Comparison of the effect of peptides from NH₂-Terminus of ANF prohormone vs. that of
atrial natriuretic factor (COOH-terminus) on sodium excretion and urine flow rate.

| | | V, μl · min⁻¹ · g | U_{Na}, | U_{Na}V μeq · min⁻¹ · g | |
|---|---|---|---|---|---|
| Peptides | n | kidney wt⁻¹ | meq/1 | kidney wt⁻¹ | FE_{NA}, % |
| Control | 9 | | | | |
| A | | 2.84 ± 0.21 | 13.36 ± 4.23 | 52.36 ± 12.05 | 0.026 ± 0.008 |
| B | | 3.37 ± 0.30 | 15.67 ± 5.26 | 59.53 ± 21/48 | 0.029 ± 0.011 |
| C | | 3.59 ± 0.40 | 13.60 ± 4.43 | 54.30 ± 19.52 | 0.023 ± 0.009 |
| D | | NS | NS | NS | NS |
| E | | NS | NS | NS | NS |
| F | | NS | NS | NS | NS |
| ProANF-(1-30) | 7 | | | | |
| A | | 1.83 ± 0.27 | 34.13 ± 10.98 | 36.37 ± 8.09 | 0.023 ± 0.002 |
| B | | 3.41 ± 0.48 | 64.83 ± 12.93 | 224.30 ± 60.34 | 0.241 ± 0.101 |
| C | | 4.31 ± 0.84 | 89.50 ± 19.82 | 384.98 ± 124.39 | 0.285 ± 0.094 |
| D | | P < 0.05 | NS | NS | NS |
| E | | P < 0.05 | P < 0.05 | P < 0.05 | P < 0.05 |
| F | | NS | NS | NS | NS |
| ProANF-(31-67) | 6 | | | | |
| A | | 2.63 ± 0.24 | 21.02 ± 4.41 | 144.08 ± 56.75 | 0.057 ± 0.012 |
| B | | 9.36 ± 3.14 | 111.73 ± 21.37 | 1,057 ± 371.69 | 0.457 ± 0.156 |
| C | | 8.84 ± 1.86 | 129.33 ± 24.73 | 1,077.63 ± 306.10 | 0.444 ± 0.155 |
| D | | P < 0.05 | P < 0.05 | P < 0.01 | P < 0.01 |
| E | | NS | P < 0.05 | P < 0.01 | P < 0.01 |
| F | | NS | NS | NS | NS |
| ProANF-(79-98) | 6 | | | | |
| A | | 2.62 ± 0.70 | 42.27 ± 24.68 | 90.88 ± 77.76 | 0.030 ± 0.024 |
| B | | 4.75 ± 1.57 | 63.30 ± 22.11 | 363.98 ± 261.85 | 0.200 ± 0.152 |
| C | | 5.16 ± 1.04 | 92.53 ± 29.17 | 564.22 ± 315.48 | 0.368 ± 0.273 |
| D | | NS | NS | NS | NS |
| E | | NS | NS | NS | NS |
| F | | NS | NS | NS | NS |
| ANF | 7 | | | | |
| A | | 2.89 ± 0.71 | 18.48 ± 3.76 | 71.77 ± 23.08 | 0.027 ± 0.010 |
| B | | 11.95 ± 4.28 | 104.07 ± 23.35 | 1,526 ± 796.50 | 0.415 ± 0.141 |
| C | | 7.26 ± 1.39 | 109.97 ± 22.12 | 838.61 ± 222.68 | 0.312 ± 0.090 |
| D | | P < 0.05 | P < 0.05 | P < 0.01 | P < 0.05 |
| E | | NS | P < 0.05 | NS | P < 0.05 |
| F | | NS | NS | NS | NS |

Values are means ± SE;
n, no. of experiments.
V, urine flow rate;
U_{Na}V, urinary sodium excretion rate;
FE_{Na}, fractional excretion of sodium,
U_{Na}, urinary sodium;
A control period;
B, first 60-minute experiment period;
C, second 60-minute experimental period;
D, ANOVA between control period and first 60-minute experimental period;
E, ANOVA between control period and second 60-minute experimental period;
F, ANOVA between first and second experimental periods.
P < 0.05 = differences between groups with ANOVA significant with 95% confidence limits.
NS = not significant.

These experiments show that three of the peptides originating from the human ANF prohormone increase both the total excretion of sodium and the fractional urine excretion of sodium. These experiments also establish increased diuresis in mammals for all of these peptides relative to the control, (Table II). Concurrently, during the course of the experiment each of the four peptides significantly (P<0.05) reduced blood pressure. Pro-ANF 79-98, the only peptide, which does not significantly increase sodium excretion was shown to be a very significant stimulator of potassium excretion (Attachment B). While ANF has been shown to significantly increase potassium excretion, pro-ANF 31-67 does not cause increased potassium excretion.

### EXAMPLE III

Testing is also carried out to determine biologic effects of these peptides by measuring their effects on particulate guanylate activity in the kidney. Human ProANF 1-30, ANF 31-67 and ANF 79-98, as well as hANF, at 1 μM concentrations, all enhance particulate guanylate cyclase from 105,000 g whole kidney homogenates, renal cortical and medullary membranes, and in the isolated distal nephrons. The human pro-ANF peptides exhibit nearly equal activity to human ANF in whole kidney homogenates and in renal medullary membranes, while in the isolated distal nephrons, some of the pro-ANF peptides are more active than human ANF itself in enhancing particulate guanylate cyclase activity, with results mirroring the renal clearance of sodium data.

### EXAMPLE IV

These peptides i.e., pro ANFs 1-30, 31-67, 79-98 can overcome the negative ionotropic (slowing heartbeat) effect of the calcium channel blocker, Verapamil. This effect suggests that these peptides may be useful for treatment of an overdose of an administered calcium channel blocker.

### EXAMPLE V

Infusion of these peptides into healthy human subjects was performed as follows: Human pro-ANF 1-30, pro-ANF 31-67, and pro-ANF 79-98, and hANF (i.e., pro-ANF 99-126, were synthesized by Peninsula Laboratories (Belmont, Calif.). High pressure liquid chromatography (HPLC) analysis was performed on a sample of each of the four peptides to ensure purity. The peptides were dissolved in a sterile 0.9% saline solution to a concentration of 100 μg/ml and dispensed in 2 and 5 ml aliquots into sealed sterile vials. At the time of dispensing, randomly selected vials were tested for pyrogens and sterility. Prior to labeling the vials, the actual peptide content of the dispensed solution were determined by direct radioimmunoassay to account for any possible absorption of the respective peptides to the walls of the vials. All vials were then stored at −80° C. until thawed for individual study.

Normal healthy subjects were chosen on the basis of age (between 20 and 50 years), physical examination and biochemical screenings. The subjects consumed their normal diets prior to testing. The subjects fasted overnight and were tested in the morning in the seated position. Intravenous catheters were placed bilaterally for the administration of pro-hANF 1-30, pro-hANF 31-67, pro-hANF 79-98 and hANF (pro-hANF 99-126) and for blood sampling. After completion of a 45-minute equilibration period, there were three 60-minute phases of the study: baseline, experimental infusion (60 minutes) and recovery (3 hours). The baseline phase consisted of two 30 minute urine collection periods, the experimental phase consisted of three 20 minute urine and plasma collection periods, and the recovery phase consisted of two 30 minute urine and plasma collection periods each hour for three hours post-infusion. Urine output was replaced on a milliliter per milliliter basis throughout the study by administering oral water or orange juice. Urine samples were obtained by voiding at the designated collection periods.

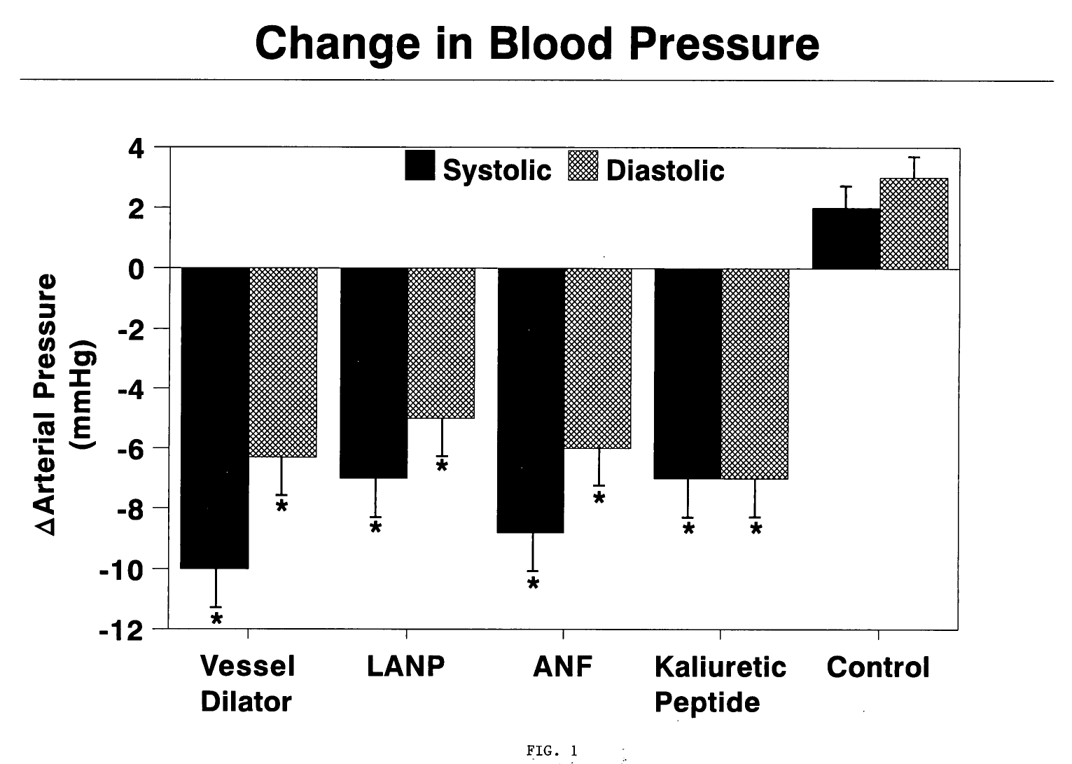

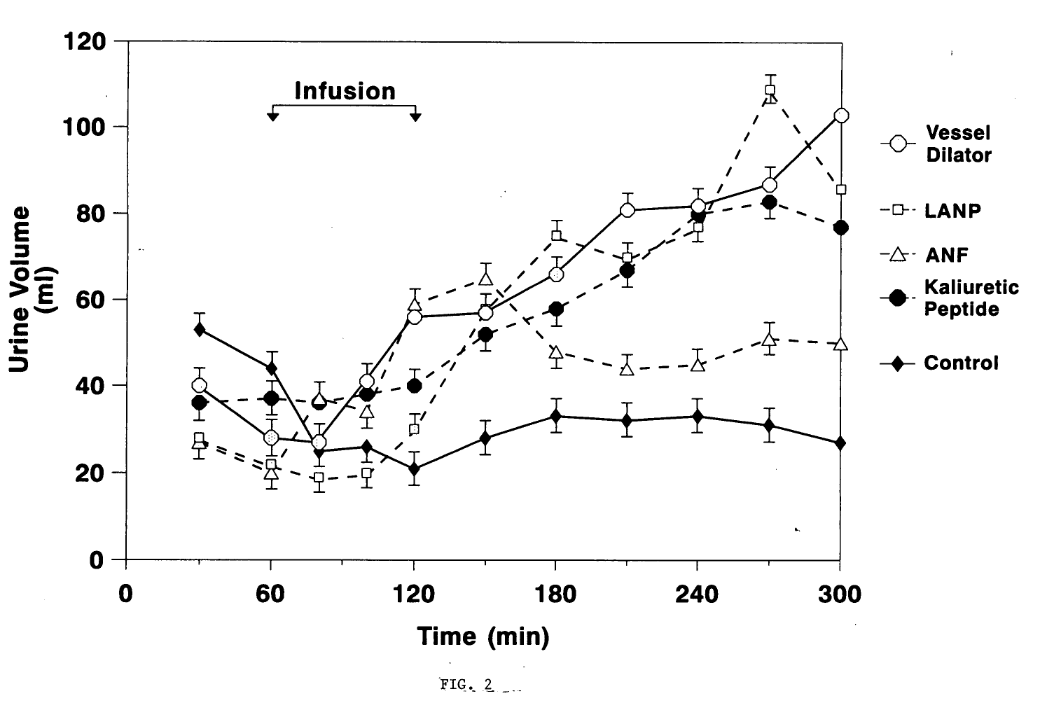

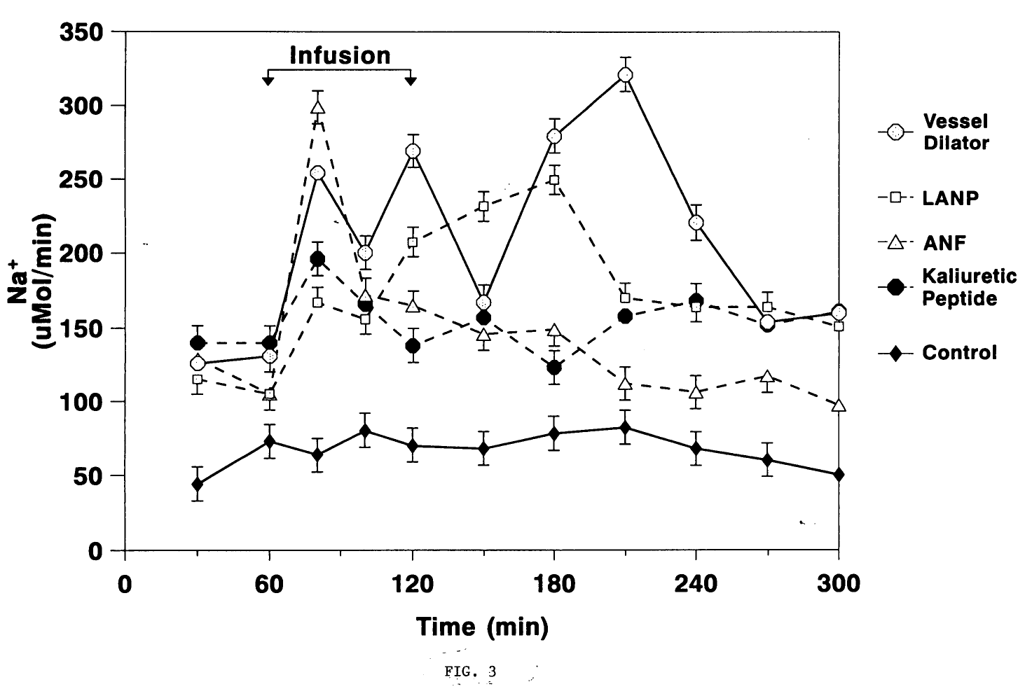

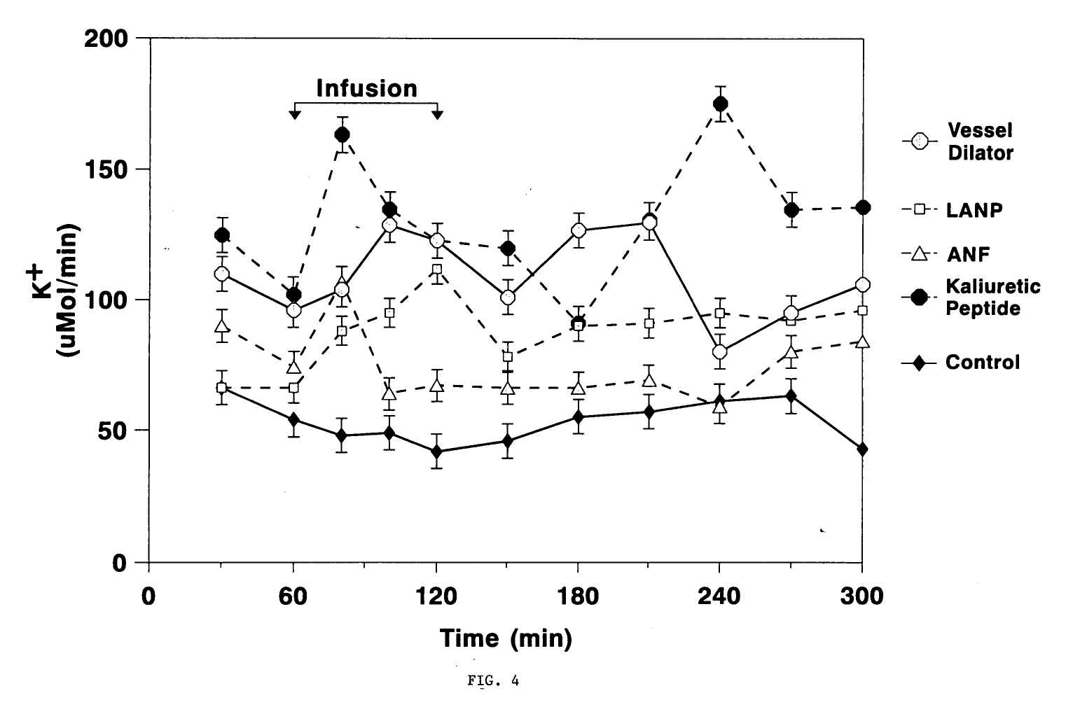

The 36 subjects were divided into 6 groups. One group (i.e., the control group) received a placebo infusion of 0.9% sodium chloride during the experimental infusion phase. While another control group received no infusion whatsoever to determine if the infusion itself has any effect. The four remaining groups of six subjects each received human pro-hANF 1-30, pro-hANF 31-67, pro-hANF 79-98 and HANF. The four groups received the peptides by continuous infusion at the rate of 0.1 μg/kg per minute. The volume administered per hour during the experimental infusion phase was 10 ml for all four peptides and the placebo. All infusions were given by a constant-rate infusion pump, with the final concentrations of prohANFs adjusted for the subjects' body weight. The subjects heart rate and cuff blood pressure were taken at 5 minute intervals throughout the study. The excreted urine was analyzed for sodium concentration. The results of the infusion study are summarized in FIGS. 1-4. The data illustrated in FIG. 1 illustrate that each of these peptides consisting of amino acid 1-30, 31-67, 79-98, and 99-126 of the human sequence of the ANF prohormone have significant blood pressure lowering effects in humans. Each of these peptides also have significant water excreting (i.e., diuretic effects) in humans (FIG. 2). As one observes in FIG. 2, ANF's diuretic effects last less than 60 minutes, while the diuretic effects of proANF 1-30, proANF 31-67, and proANF 79-98 are still significant in humans 3 hours after stopping their respective infusions. This ability to cause a diuresis significantly (P<0.001) longer than ANF is a property that causes these peptides to be preferred over ANF in the treatment of water retaining states, as demonstrated in example VI of humans with congestive heart failure. ProANF 1-30 and proANF 31-67 have significant natriuretic (i.e., sodium excreting) properties in humans that last significantly (P<0.001) longer than ANF's effects (FIG. 3). ProANF 79-98 was found to have the property of being a significant (P<0.01) enhancer of potassium excretion in humans (FIG. 4).

### EXAMPLE VI

Human proANF's 1-30, 31-67, 79-98, and 99-126 (i.e., ANF) were infused into human subjects with Class III New York Heart Association congestive heart failure (CHF). An identical protocol to that used in example V where these peptides were infused into healthy human subjects was utilized. (See Attachment C and F for a detailed description of this protocol). The human subjects with congestive heart failure received an identical dose (0.1 μg/kg body wt/minute for 60 minutes) that the healthy human subjects received. With infusion of proANF 31-67, urine output doubled in 20 minutes and increased to 4.8-fold at the end of the 60 minute infusion (Vesely D L, et al., Circulation 98:323-329, 1998). The urine output continued to increase after stopping the infusion of pro ANF 31-67 and at 3 hours after stopping the infusion the urine output of the individuals with Class III congestive heart failure was 4.3-fold that of pre-infusion values (i.e., 6.7±0.65 mL/min versus 1.56±0.35 mL/min at baseline). This striking urine output secondary to proANF 31-67 was significant at P<0.0001 compared to the pre-infusion output of the congestive heart failure subjects (n=6). One patient who was studied for a longer period exhibited a 2-fold increase in urinary volume and flow for six hours after proANF 31-67 infusion was stopped. ProANF 31-67 also enhanced sodium excretion 3- to 4-fold (P<0.01). It caused a doubling of sodium excretion within 20 minutes; and 3 hours after the proANF 31-67 infusion was stopped, sodium excretion was still 3-fold greater than baseline sodium excretion (P<0.01) (ibid).

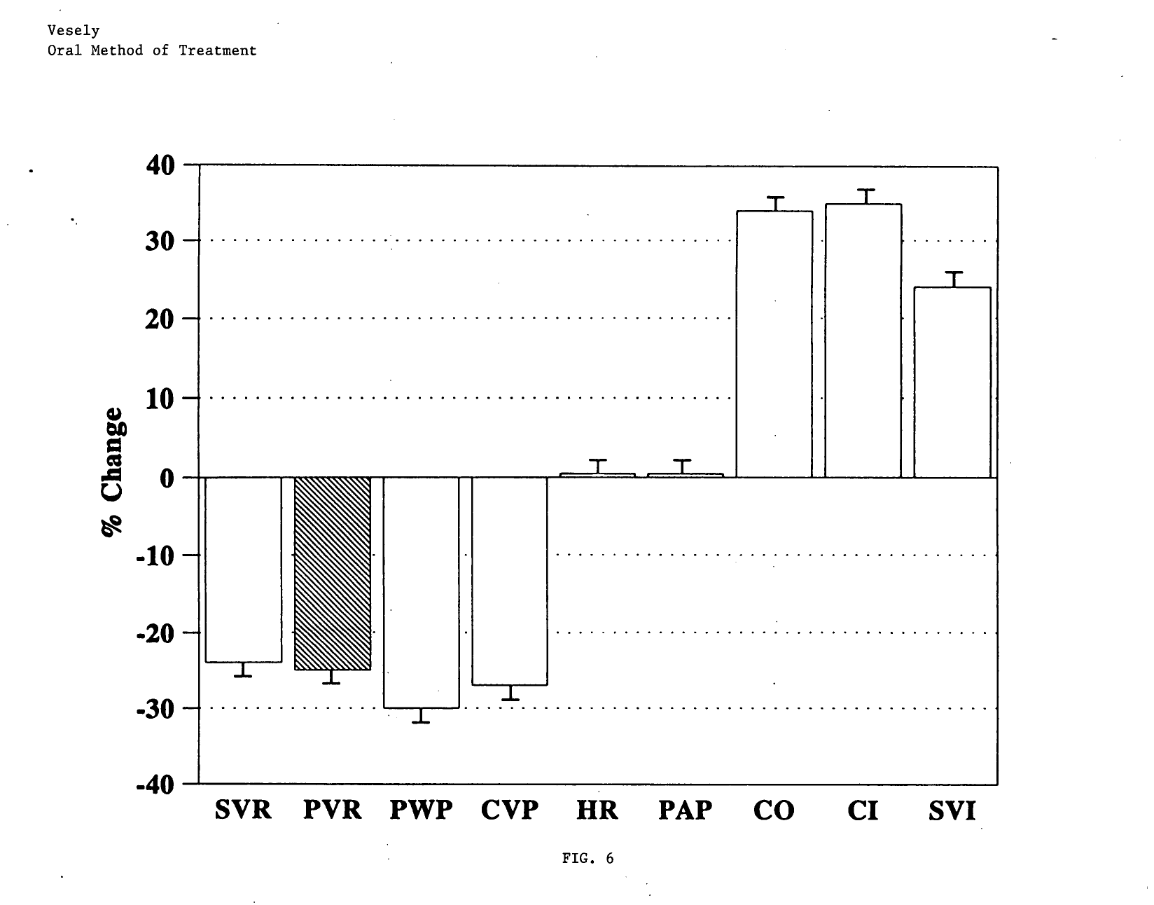

ProANF 31-67 increased fractional excretion of sodium (FENa) in patients with CHF up to a maximum of 6-fold (P<0.001) (ibid). Thus, the FENa doubled 20 minutes after proANF 31-67 infusion began, and it was 4.5-fold greater than baseline at the end of the infusion. During the infusion, systemic vascular resistance decreased 24% and pulmonary vascular resistance decreased 25% (FIG. 6) (ibid). Pulmonary capillary wedge pressure decreased 33% and central venous pressure decreased 27%. Heart rate and mean pulmonary artery pressure did not change significantly; however, proANF 31-67 increased cardiac output 34% (5.35±0.9 to 7.9±1.2 L/min) and cardiac index 35% (2.66±0.01to 3.58±0.01 L·min⁻¹·m⁻²) (ibid). It also increased stroke volume index by 24%. There were no adverse effects associated with the use of proANF 31-67.

Pro ANF's 1-30, likewise, caused a significant diuresis in the congestive heart failure individuals (n=6 for each group) with urine output increasing 2- to 6-fold at the end of its infusion and the urine flow secondary to these peptides still be elevated (P<0.01) 3 hours after stopping their infusions (Vesely D L, et al. Am. Heart J. 138:625-632, 1999; Attachment G). ProANF 1-30 increased the fractional excretion of sodium 3-fold in the congestive heart failure individuals (ibid). The natriuretic and diuretic effects in Class III CHF individuals, however, were not as significant as their effects in healthy persons (ibid). ProANF 79-98 effects are not blunted in persons with CHF but rather are increased compared to healthy individuals (Nasser A, et al. Am. J. Cardiol. 88:23-29, 2001). ProANF 79-98 (100 ng/kg body weight/min) given intravenously for 60 minutes to subjects with New York Heart Association class III CHF increased urine flow 4-fold (P<0.001), which was maximal 2±hours after its infusion was stopped, compared to a 2-fold increase in urine flow in healthy adults (ibid). ProANF 79-98 enhanced sodium excretion 3-fold in subjects with CHF (P<0.01). ProANF 79-98 enhancement of sodium excretion in CHF subjects is a feature not observed in persons without sodium retention, i.e., healthy adults (ibid).

ANF's effects, on the other hand, were blunted in the human subjects with congestive heart failure. ANP (same concentration) did not significantly enhance urine flow. ANP enhanced sodium excretion 2- to 6-fold in half of the CHF subjects, whereas it had no effect on sodium excretion in the other half. ANP did not significantly increase fractional excretion of sodium but did increase fractional excretion of potassium (P<0.05) during the first 20 minutes of its infusion. ANP-infused patients with CHF became hypotensive. None became hypotensive secondary to proANF 1-30, 31-67 or 79-98. Thus, proANFs 1-30, 31-67, and 79-98 have stronger diuretic and natriuretic properties than ANF in congestive heart failure patients. These peptides' effects on producing a diuresis whereby they were still markedly enhancing urine flow 3 hours after their infusions had ceased indicate that they definitely have utility in treating humans with congestive heart failure.

Sodium excretion also increased in the human subjects with congestive heart failure three-to-eight fold with proANF 1-30 and 31-67 while ANF's effects on sodium excretion was blunted in persons with congestive heart failure compared to a 4- to 11-fold increase in healthy humans (ibid). This series of investigations demonstrates that proANF 1-30, proANF 31-67 and proANF 79-98 have utility as treatment of congestive heart failure in humans with strong diuretic and sodium excreting properties in persons with congestive heart failure. One skilled in the art of medicine can, thus, foresee that proANF 1-30 and/or proANF 31-67 may have proANF 79-98 added sequentially as part of a treatment regimen of persons with congestive heart failure (and other sodium and water retaining states) to help maintain their sodium and water balance.

These investigations in human subjects with congestive heart failure demonstrate the utility of the human forms proANF 1-30, proANF 31-67 and proANF 79-98 in the most common disease (i.e., congestive heart failure) characterized by sodium and water retention. One would anticipate that these peptides would also be useful in other less common forms of salt and water retention (delineated above) since their therapeutic effects would be the same in all diseases characterized by sodium and water retention, i.e., their therapeutic benefit derives from their ability to increase sodium and water excretion in humans and other mammals. This has been proved-in-practice for acute renal failure (Example VII).

The data in Example V (which includes FIGS. 1-4) demonstrate that the human forms proANFs 1-30, 31-67, and 79-98 have similar effects in humans as they do in other mammals. In healthy humans proANF 31-67 lowers blood pressure the most and one would, thus, discern that it may be the most useful therapeutically for lowering blood pressure in humans. It is important to note that each of the above peptides lower blood pressure in humans and therefore using one or more of these peptides in combination may have added therapeutic benefit as antihypertensive agents.

Example VI demonstrates that proANFs 1-30, 31-67 and 79-98 have therapeutic benefit in humans with congestive heart failure. Since these peptides work similarly in all diseases characterized by sodium and water retention by increasing sodium and water excretion, one would expect that each of these peptides will work in all human (and mammals) diseases characterized by sodium and water retention. In the case of acute renal failure (i.e., acute tubular necrosis) this has been brought to practice (Example VII). Even when acute tubular necrosis has been present for two days when one gives proANF 31-67 there is a dramatic improvement in the renal failure and the tubules regenerate resulting in a markedly improvement mortality in this condition which can be lethal (Clark L C, et al. Am. J. Physiol. 278:H1555-H1564, 2000).

### EXAMPLE VII

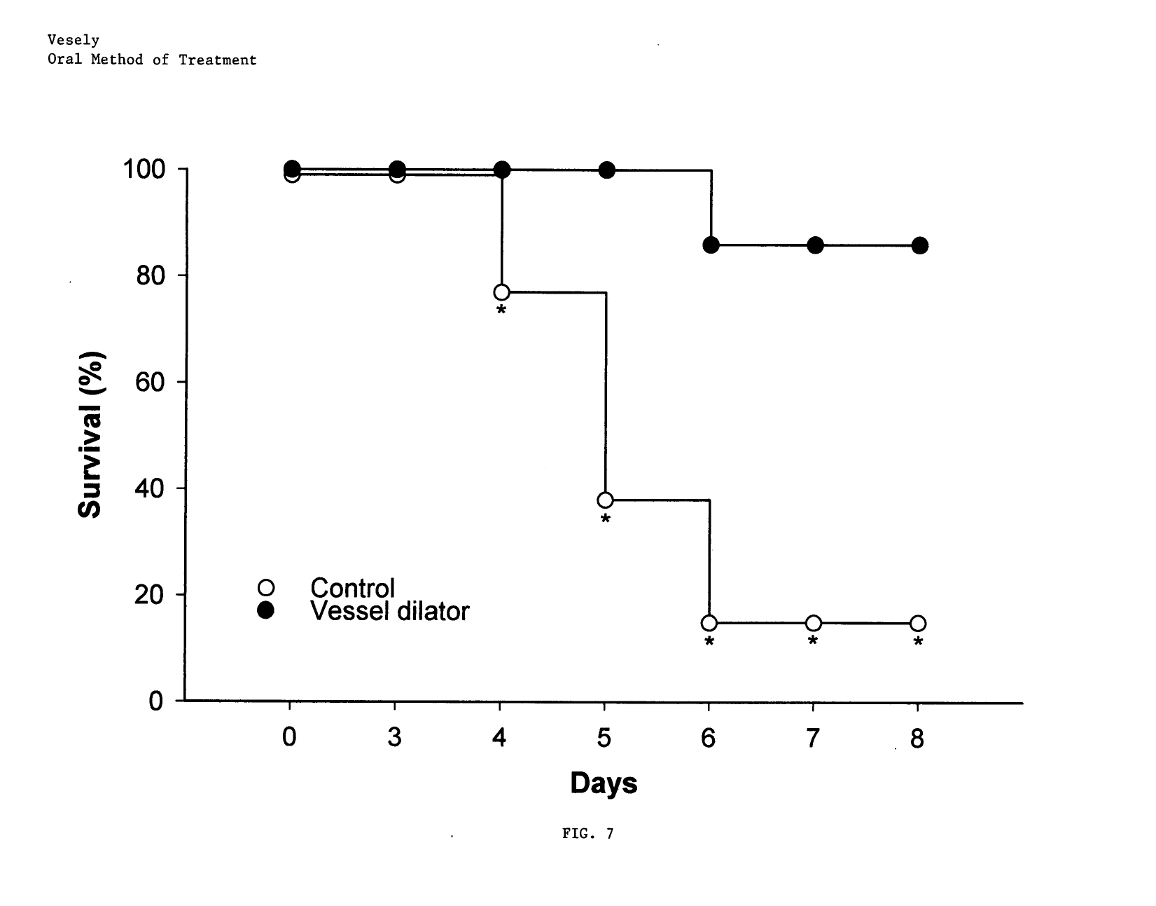

Ischemic nonologuric acute renal failure (ARF) was induced in 24 male Sprague-Dawley rats (Zivc-Miller), weighing 200-270 g with 50 min of ischemia. Seven of the ARF rats received proANF 31-67. There were 17 ARF rats in the control group. Each of these rats were handled identically with the control rats as well as the experimental group of rats receiving an osmotic pump placement two days after receiving a unilateral right nephrectomy followed by clamping left renal artery for 50 min. The only difference was that the control group received 0.9% saline only in their osmotic pumps, whereas the experimental group had proANF 31-67 dissolved in 0.9% saline within their osmotic pumps which pump for 72 hours into the peritoneal cavity. In the 17 control ARF rats their preischemic blood urea nitrogen (BUN) and creatinine levels of 16±1 and 0.56±0.05 mg/dl increased to 162±4 and 8.17±0.5 mg/dl, P<0.001, respect 4 of postischemia. ProANF 31-67 (0.3 μg·kg⁻¹·min⁻¹ intraperitoneally), decreased the BUN and creatinine levels to 53±17 mg/dl and 0.98±0.12 mg/dl (P<0.001) in seven animals where ARF had been established for two days. Water excretion doubled with ARF and was further augmented by proANF 31-67. Transthoracic echocardiography revealed left ventricular dilation as a probable cause of the increase in proANF 31-67 in the circulation with ARF, and proANF 31-67 infusion reversed this dilation, At day 6 of ARF, mortality decreased to 14% with proANF 31-67 from 88% without proANF 31-67 (FIG. 7). Acute tubular necrosis was <5% in the proANF 31-67-treated rats compared with 25% to >75% in the placebo-treated ARF animals.

Thus, any one of the three peptide fragments or a non-toxic salt thereof, combined with a pharmaceutically acceptable carrier to form a pharmaceutical composition, may be administered to mammals, including humans, orally. The peptide(s) should be at least about 95% pure and preferably should have a purity of 100%. This purity means that the intended peptide constitutes the stated weight (%) of all like peptides and peptide present. Administration should be prescribed by a physician for humans and by a veterinarian for all other mammals, and the dosage will vary with the particular condition being treated. It may be desirable to administer combinations of two of the three peptides or to administer all three peptides, either simultaneously or in separate preparations, to take advantage of the different biologic effects and potencies of the three peptides in an effective, synergistic therapeutic treatment.

Peptides similar to proANF 1-30, proANF 31-67, and proANF 79-98 are often administered in the form of pharmaceutically acceptable non-toxic salts, such as acid addition salts or metal complexes, e.g., with zinc, iron, calcium, barium, magnesium, aluminum or the like (which are considered as addition salts for purposes of this application). Illustrative of such acid addition salts are hydrochloride, hydrobromide, sulphate, phosphate, tannate, oxalate, fumarate, gluconate, alginate, maleate, acetate, citrate, benzoate, succinate, malate, ascorbate, tartrate and the like.

The peptides should be administered to humans under the guidance of a physician, and pharmaceutical compositions will usually contain the peptide in conjunction with a conventional, pharmaceutically-acceptable carrier. The dosage may be from about 0.1 to about 200 micrograms of the peptide per kilogram of the body weight of the host depending on the specific condition being treated.

Oral Methods of Treatment using ProANF Peptides:

This application builds upon my U.S. Pat. No. 5,691,310 to add a new method(s) to give these peptides i.e., “orally” [by mouth]. It has now been found that peptide hormones similar to proANFs 1-30, 31-67 and 79-98 in the above United States Patent can be given orally by several different methods that were not available when the above patent was granted.

Examples of Different Technologies that Allow for these Peptides to be given Orally:

### EXAMPLE I

One of the atrial natriuretic peptides, i.e., the class that above three peptide hormones belong to, has been given orally by attaching amiphiphillic oligomers covalently attached to peptides to protect them from proteoloysis in the gastrointestinal tract and found to have the biologic effect of lowering blood pressure (Cataliotti A, et al. J. Am. Coll. Cardiol. 45(3 Supplement A): p 419A, 2005 [Abstract]). This biologic effect suggests that the proteases in the gastrointestinal tract did not degrade this peptide.

### EXAMPLE II

In addition to the above method another method to give these peptides orally is replacing one of the 1-isomer amino acids at either end of the peptide with a d-isomer so the peptide is not degraded with proteases as it enters the gastrointestinal tract. All proteins when synthesized by the body, such as proANFs 1-30, 31-67, and 79-98, contain amino acids which are 1-isomers. All enzymes in the body which break down peptides recognize and degrade the 1-isomer forms of amino acids within peptides. By replacing one or more of the 1-isomer amino acids with a d-isomer amino acid near the end of the peptide chain results in the inability of enzyme (proteases) to recognize and degrade peptides. One peptide, i.e., bradykinin has been demonstrated to be protected from proteases with this method when given orally. Some of the atrial natriuretic peptides are degraded by the same proteases that decrease bradykinin (Vesely D L. Atrial Natriuretic Peptides, Englewood Cliffs, N.J.; Prentice Hall; 1992; pp 1-256) so this would be another method for at least some of the atrial natriuretic peptides to give them orally.

### EXAMPLE III

A third method to give peptides orally such as the above peptides in the U.S. Pat. No. 5,691,310 is nanotechnology including but not limited to chitosan as a drug delivery system. Chitosan is a linear amino polysaccharide of glucosamine and N-acetylglucosamine, and can be derived by partial N-deacetylation of chitin from crustacean shells. One of the key features of chitosans is that upon dissolution, the amine groups of chitosan molecules are protonated and the resultant soluble polymer is positively charged. Chitosan is degraded by lysozymes in the body into a common amino sugar, N-acetyl glucosamine, which is incorporated into the synthetic pathway of glycoproteins and excreted as a carbon dioxide (Lee M N, et al. Pharmaceutical Research 18:427-431, 2001).

The chemical reaction of chitosan occurs primarily with the free amine groups. Thiolated chitosans, which appear more useful than unthiolated chitosan for complexing with peptides, are obtained by conjugating thiol group moieties to free amine groups through an amide bond. Some free amine groups on chitosan participate in the chemical reaction. The remaining free amine groups, when dissolved, are protonated to get positively charged and consequently participate in the particle formation by ionic cross-linking with multianions. The above peptides attached chitosan molecules can be delivered orally in liposomes. Liposomes are vesicles formed by concentric spherical phospholipids bilayers encapsulating an aqueous space. The inner aqueous compartment can be used for encapsulating peptides or proteins with a higher degree of protection against enzymatic degradation. The protonated amine groups readily associate with the negatively charged phospholipids and subsequently enable them to coat the liposome surface. The positive charge contributed from the protonated amine groups also allows the chitosan nanoparticles and chitosan-coated liposomes to come into closer association with the negatively charged cell membrane (Mao H Q, et al. J. Controlled Release 70:399-421, 2001).

What is claimed is:

1. A method of treating high blood pressure, congestive heart failure, pulmonary edema, toxemia of pregnancy, nephrotic syndrome, acute and chronic renal failure, hepatic cirrhosis with and without ascites; which method comprises administering to a mammal, including humans, a therapeutically effective amount of at least 95% pure synthetic peptide from the group of peptides consisting of amino acids 1-30 (i.e., proANF 1-30), amino acids 31-67 (i.e., proANF 31-67), and amino acids 79-98 (i.e., proANF 79-98) of the human sequence of the 126 amino acid atrial natriuretic factor prohormone; which have blood pressure lowering, water, sodium and/or potassium excreting properties in humans; and combinations thereof or a non-toxic salt thereof.

2. A method according to claim 1 wherein said peptide (proANF 1-30) has the following sequence: H-Asn-Pro-Met-Tyr-Asn-Ala-Val-Ser-Asn-Ala-Asp-Leu-Met-Asp-Phe-Lys-Asn-Leu-Leu-Asp-His-Leu-Glu-Glu-Lys-Met-Pro-Leu-Glu-Asp-OH, synthesized with an automated peptide synthesizer or other in vitro peptide synthesis techniques.

3. A method according to claim 1 wherein said peptide (proANF 31-67) has the sequence: H- Glu-Val-Val-Pro Pro-Gln-Val-Leu-Ser-Glu-Pro-Asn-Glu-Glu-Ala-Gly-Ala-Ala-Leu-Ser-Pro- Leu-Pro-Glu-Val-Pro-Pro-Trp-Thr-Gly-Glu-Val-Ser-Pro-Ala-Gln-Arg-OH, synthesized with an automated peptide syntesizer or other in vitro peptide synthesis techniques.

4. A method according to claim 1 wherein said peptide (proANF 79-98) has the sequence: H-Ser- Ser-Asp-Arg-Ser-Ala-Leu-Leu-Lys-Ser-Lys-Leu-Arg-Ala-Leu-Leu-Thr-Ala-Pro-Arg-OH, synthesized with an automated peptide synthesizer or other in vitro synthesis techniques.

5. A method according to claim 1 wherein said peptide(s) being of at least 95% pure or a non-toxic salt thereof is administered in a pharmaceutically acceptable liquid or solid carrier.

6. A method in accordance with claim 1 wherein said administration is carried out orally.

7. A method in accordance with claim 2 wherein said administration is carried out orally.

8. A method in accordance with claim 3 wherein said administration is carried out orally.

9. A method in accordance with claim 4 wherein said administration is carried out orally.

10. A method of claim 1 wherein said administration is at a level between about 0.1 and 200 micrograms per kilogram of body weight.

11. A method of claim 2 wherein said administration is at a level between about 0.1 and 200 micrograms per kilogram of body weight.

12. A method of claim 3 wherein said administration is at a level between about 0.1 and 200 micrograms per kilogram of body weight.

13. A method of claim 4 wherein said administration is at a level between about 0.1 and 200 micrograms per kilogram of body weight.

14. An alternate method in accordance with claim 1 wherein a combination of at least two of said peptides is administered.

15. An alternate method in accordance with claim 1 wherein proANF 1-30 is administered in combination with proANF 31-67.

16. An alternate method in accordance with claim 1 wherein proANF 1-30 is administered in combination with proANF 79-98.

17. An alternate method in accordance with claim 1 wherein proANF 31-67 is administered in combination with proANF 79-98.

18. An alternate method in accordance with claim 1 wherein proANF 1-30, proANF 31-67, and proANF 79-98 are administered together.

19. A pharmaceutical composition for treating high blood pressure, congestive heart failure, nephrotic syndrome, acute and chronic renal failure, toxemia of pregnancy, pulmonary edema, and hepatic cirrhosis, which composition consists essentially of a therapeutically effective amount of at least 98% pure synthetic human peptide which is selected from the group of proANF 1-30, proANF 31-67, proANF 79-98 and a combination thereof or non toxic salts thereof, plus a pharmaceutically acceptable liquid or solid carrier given orally.

20. A composition according to claim 19 wherein said peptide (proANF 1-30) has the following sequence: H-Asn-Pro-Met-Tyr-Asn-Ala-Val-Ser-Asn-Ala-Asp-Leu-Met-Asp-Phe-Lys-Asn-Leu-Leu-Asp-His-Leu-Glu-Glu-Lys-Met-Pro-Leu-Glu-Asp-OH.

21. A composition according to claim 19 wherein said peptide (proANF 31-67) has the sequence: H-Glu-Val-Val-Pro-Pro-Gln-Val-Leu-Ser-Glu-Pro-Asn-Glu-Glu-Ala-Gly-Ala-Ala-Leu-Ser-Pro- Leu-Pro-Glu-Val-Pro-Pro-Trp-Thr-Gly-Glu-Val-Ser-Pro-Ala-Gln-Arg-OH.

22. A composition according to claim 19 wherein said peptide (proANF 79-98) has the sequence: H-Ser-Ser-Asp-Arg-Ser-Ala-Leu-Leu-Lys-Ser-Lys-Leu-Arg-Ala-Leu-Leu-Thr-Ala-Pro-Arg-OH.

23. A oral method of treating sodium and water retaining conditions which include congestive heart failure, renal failure, toxemia of pregnancy, and hepatic cirrhosis with ascites comprising administering to a human a therapeutically effective amount of a 100% pure synthetic peptide from the group consisting of the human sequences proANF 1-30, proANF 31-67, proANF 79-98 and combinations thereof or a non-toxic salt thereof.

24. Oral method(s) of regulating hypertension comprising administering to a human a therapeutically effective amount of at least 95% pure, synthetic peptide from the group of peptides consisting of the human sequenced proANF 1-30, proANF 31-67, proANF 79-98 and a combination thereof or a non-toxic salt thereof.

25. Oral method(s) of treating hyperkalemia (i.e., high blood potassium) associated with acidosis, digitalis overdosage, succinylcholine, insulin deficiency, acute and chronic renal failure, Addison's disease (i.e., adrenal insufficiency) and the hypoaldosteronism comprised of administering to humans and other mammals a therapeutically effective amount of proANF 79-98 or proANF 1-30 or a combination thereof or non-toxic salt thereof.
